(19) United States
(12) Patent Application Publication
(10) Pub. No.: US 2024/0173089 A1
(43) Pub. Date: May 30, 2024

(54) ROBOTIC REMOTE CENTER OF MOTION WITH ACTIVE PORTAL/TROCAR MANIPULATION

(71) Applicant: Covidien LP, Mansfield, MA (US)

(72) Inventors: Ranjan Kumar Mishra, Orange, CT (US); Vamsi Krishna Chaitanya Molugu, Branford, CT (US); Eric J. Taylor, Southington, CT (US)

(21) Appl. No.: 18/281,949

(22) PCT Filed: Apr. 14, 2022
(86) PCT No.: PCT/US2022/024742

§ 371 Date: Sep. 13, 2023

Related U.S. Application Data
(60) Provisional application No. 63/174,735, filed on Apr. 14, 2021.

(51) Int. Cl. A61B 34/35

(52) U.S. Cl. CPC A61B 34/35

(57) ABSTRACT

A robotic surgical system includes a robotic arm, a surgical instrument, a surgical portal, and a linear slide mechanism. The linear slide mechanism is coupled to the robotic arm and is configured to movably support the surgical portal relative to the surgical instrument. The linear mechanism can support a movable carriage and a movable rail positioned to translate relative to moveable carriage to enable a surgical instrument to move relative to movable carriage.

### CROSS REFERENCE TO RELATED APPLICATIONS

This application is a 371 National Stage Application of International Application No. PCT/US2022/024742, filed Apr. 14, 2022, which claims the benefit of U.S. Provisional Patent Application Ser. No. 63/174,735, filed Apr. 14, 2021, the entire contents of which are incorporated by reference herein.

### BACKGROUND

Robotic surgical systems have been used in minimally invasive medical procedures in which surgical instruments were inserted through surgical portals at fixed entry points into the patient's body. These systems incorporated a Remote Center of Motion (RCM) to ensure that the surgical instruments did not move beyond these fixed entry points as the instruments were manipulated inside the patient's body. Many of these surgical robots used a mechanical RCM with a portion of the robotic arm attaching directly to the surgical portal. Unlike surgical robots using mechanical RCMs, software-based RCMs typically did not mechanically connect to the surgical portal in order to provide an increased range of motion and reduce collisions between the robotic arms of the surgical robot. Unfortunately, many of the surgical robots with software-based RCMs complicated instrument exchanges as the surgical portals moved out of alignment with the robotic arms when the surgical instruments were removed.

During an instrument exchange, the surgical instrument was pulled out of the surgical port and removed from the robotic arm. A new or different surgical instrument was then connected to the robotic arm and introduced back through the surgical portal. Surgical robots with mechanical based RCMs facilitated the exchange because the surgical portal was continually held in alignment with the linear axis of the instrument motion by a linkage or connection to the surgical portal. In contrast, surgical robots with software-based RCMs did not have a connection or linkage to the surgical portal and therefore lost alignment when the surgical instrument was removed from the surgical portal. Inserting another surgical instrument required the clinician to manually align the surgical portal with the surgical instrument. This process increased the time required for conducting the instrument exchange.

Indeed, compared to mechanical RCMs, software-based RCMs had the following limitations: free-floating surgical portals were supported by the patient through surrounding tissue and during instrument exchange; such surgical portals provided a contact surface to the surgical instrument and lateral forces that acted on such surgical portals were absorbed by patient tissue; and there was an indirect software level joint torque control that provided constraints to prevent movement of the surgical portal within the incision point.

### SUMMARY

According to aspects, this disclosure is directed to a robotic surgical system including a direct mechanical connection to a surgical portal and which addresses the above-noted software-based RCM limitations with at least the following solutions: a seven (7) degree of freedom manipulator; a one (1) degree of freedom surgical portal extension or retraction (active or passive); an electronic gearing and active joint drive integrated with a brake to provide controlled gravity compensations and power off lock with on demand manual override; a six (6) degree of freedom force/torque sensor on a surgical portal mount assembly that provides reaction force-torque data from the patient incision point; an independent 1-axis instrument roll; and an independent multi-axis instrument drive unit.

According to one aspect, this disclosure is directed to a robotic surgical system. The robotic surgical system includes a robotic arm, a surgical instrument, a surgical portal, and a linear slide mechanism. The linear slide mechanism is coupled to the robotic arm and configured to movably support the surgical portal relative to the surgical instrument.

In aspects, the linear slide mechanism may include an arm assembly including a stationary segment and a movable segment. The movable segment may be positioned to move relative to the stationary segment. The movable segment may slide along the stationary segment. The movable segment may be movable relative to the surgical instrument.

In aspects, the linear slide mechanism may include a drive housing on a proximal end portion thereof. The drive housing may be coupled to the robotic arm. The stationary segment may extend from the drive housing. The drive housing may be coupled to an instrument drive unit that operates the surgical instrument.

In aspects, the robotic surgical system may further include a sterile interface module that connects the surgical instrument to the instrument drive unit.

In aspects, the movable segment may support a mount assembly that is configured to support the surgical portal. This eliminates the need for an instrument to support the surgical portal and prevents side loads on the instrument shafts to sense and monitor software RCM.

In aspects, the drive housing may support a drive motor and a drive that is operable by the drive motor to cause the movable segment to move relative to the stationary segment. The drive may include a cable drive, a belt drive, a rack and pinion drive, linear electro-magnetic motor or combinations thereof integrated with either linear or rotary position sensor/encoder to provide linear displacement of the movable segment relative to the stationary segment to achieve servo position control and force control.

According to one aspect, this disclosure is directed to a surgical system. The surgical system includes an instrument drive unit, a surgical instrument coupled to the instrument drive unit, a surgical portal, and a linear slide mechanism configured to movably support the surgical portal relative to the surgical instrument.

In aspects, the linear slide mechanism may include a drive housing on a proximal end portion thereof and the drive housing may be coupled to the instrument drive unit.

In aspects, the drive housing may support an encoder configured to monitor positions of the movable segment relative to the stationary segment. The drive housing may include a rotary encoder at drive motor, a cable pulley, a belt pulley, a pinion gear or linear encoder at the movable segments, a belt, a cable, a linear motor, or combinations thereof.

In aspects, the surgical system may further include a sterile interface module that connects the surgical instrument to the instrument drive unit.

According to one aspect, the robotic surgical system includes a robotic arm, an instrument drive unit, a surgical instrument removably coupled to the instrument drive unit, a surgical portal, a controller, and a linear slide mechanism positioned between the robotic arm and the instrument drive unit. The linear slide mechanism includes a movable segment that is operatively coupled to the controller to enable the controller to actively move the surgical portal relative to a patient.

Other aspects, features, and advantages will be apparent from the description, the drawings, and the claims that follow.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the aspects given below, explain the principles of the disclosure, wherein:

FIG. 1 is a schematic view of a robotic surgical system in accordance with the principles of the present disclosure;

FIG. 2 is a perspective view of a robotic arm of the robotic surgical system of FIG. 1, the robotic arm shown with a surgical assembly coupled thereto, the surgical assembly having a surgical instrument, a linear slide mechanism, and a surgical portal coupled thereto;

FIG. 3 is an enlarged, perspective view of the surgical assembly of FIG. 2 with a different surgical instrument coupled thereto and with the linear slide mechanism thereof shown in a first, extended position;

FIG. 4 is another view of FIG. 3 with the linear slide mechanism shown in a second, retracted position;

FIG. 5 is an enlarged view of the indicated area of detail shown in FIG. 3;

FIG. 6 is another view of FIG. 5 illustrating the surgical portal separated from a mount assembly of the surgical assembly of the robotic surgical system of FIG. 1;

FIG. 7 is another perspective view illustrating the linear slide mechanism actively manipulating the surgical portal when the surgical instrument of FIG. 3 is advanced through the surgical portal for effectuating a surgical procedure on a patient;

FIG. 8 is an enlarged, cross-sectional view as taken along section line 8-8 shown in FIG. 7; and

FIG. 9 is a perspective view of another surgical assembly of the robotic surgical system of FIG. 1.

### DETAILED DESCRIPTION

Aspects of this disclosure are described in detail with reference to the drawings, in which like reference numerals designate identical or corresponding elements in each of the several views. As used herein, the term “distal” refers to that portion of a device that is farther from the user, while the term “proximal” refers to that portion of a device that is closer to the user. As used herein, the term “clinician” refers to a doctor, nurse, or other care provider and may include support personnel.

In the following description, well-known functions or constructions are not described in detail to avoid obscuring the present disclosure in unnecessary detail.

Robotic surgical systems have been used in minimally invasive medical procedures and can include robotic arm assemblies. Such procedures may be referred to as what is commonly referred to as “Telesurgery.” Some robotic arm assemblies include one or more robot arms to which surgical instruments can be coupled. These surgical instruments include, for example, electrosurgical forceps, cutting instruments, staplers, graspers, electrocautery devices, or any other endoscopic or open surgical devices. Prior to or during use of the robotic surgical system, various surgical instruments can be selected and connected to the robot arms for selectively actuating end effectors of the connected surgical instruments. Such systems employ various robotic elements to assist the clinician and allow remote operation (or partial remote operation) of surgical instrumentation. Various gears, cams, pulleys, electric and mechanical motors, etc. may be employed for this purpose and may be designed with a robotic surgical system to assist the clinician during the course of an operation or treatment. These robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of clinicians may prep the patient for surgery and configure the robotic surgical system with one or more of the instruments disclosed herein while another clinician (or group of clinicians) remotely controls the instruments via the robotic surgical system. As can be appreciated, a highly skilled clinician may perform multiple operations in multiple locations without leaving his/her remote console which can be both economically advantageous and a benefit to the patient or a series of patients.

This disclosure details a robotic surgical system that provides at least one of the benefits of: direct mechanical connection of the robotic surgical system to a surgical portal that receives the surgical instruments therethrough (e.g., in sealed relationship therewith); active manipulation of the surgical portal in robotic minimally invasive surgery using software RCM; direct force/torque sensing at the incision point that provides direct feedback for controlling and maintaining RCM at the desired incision point making the current software based force/torque sensing as a redundant feedback; provide significant patient safety by supporting the portal/trocar and preventing free floating during instrument exchange, robo-lift to increase a clinician's access; and additional joint redundancy that enables dynamic collision avoidance, optimized robot placement, and improved bed-side access. Further, the disclosed robotic surgical system can hold the surgical portal with an instrument attached and provide safe manipulation of the surgical portal to optimize instrument reachability for safe and efficient manipulation inside the patient cavity with robo-lift.

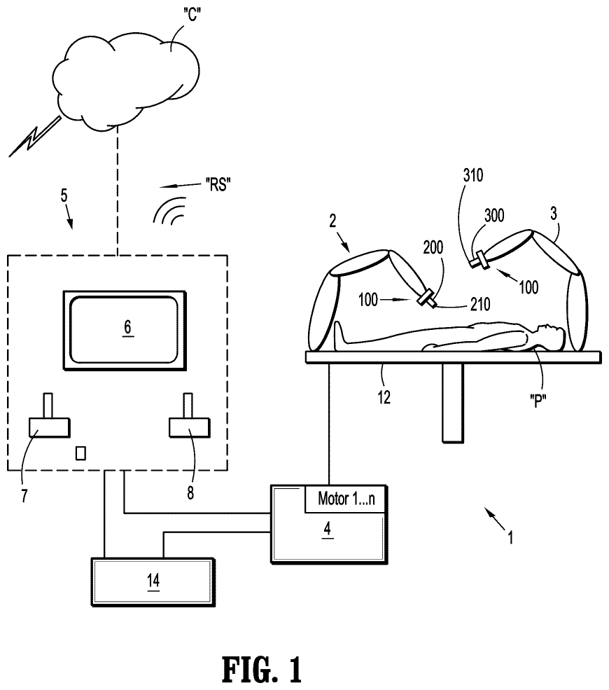

Referring initially to FIG. 1, a surgical system, such as, for example, a robotic surgical system is shown generally as robotic surgical system 1 and generally includes a plurality of robotic arms 2, 3; a controller or control device 4; and an operating console 5 coupled with control device 4. Operating console 5 includes a display device 6, which is set up in particular to display three-dimensional images; and manual input devices 7, 8, by means of which a person (not shown), for example a surgeon, is able to telemanipulate robotic arms 2, 3 in a first operating mode, as known in principle to a person skilled in the art.

Robotic surgical system 1 also includes a surgical assembly 100 connected to a distal end of each of robotic arms 2, 3. Surgical assembly 100 may support one or more surgical instruments such as surgical instruments 200, 300 (e.g., a grasper, stapler, cutter, sealer, or the like).

Each of the robotic arms 2, 3 is composed of a plurality of members, which are connected through joints. Robotic arms 2, 3 may be driven by electric drives (not shown) that are connected to control device 4. Control device 4 (e.g., a computer) is set up to activate the drives, in particular by means of a computer program, in such a way that robotic arms 2, 3, their surgical assemblies 100 and/or surgical instruments 200, 300 execute a desired movement according to a movement defined by means of manual input devices 7, 8. Control device 4 may also be set up in such a way that it regulates movement of robotic arms 2, 3 and/or of the drives. While electrically coupled to controller or control device 4, as described above, robotic arms 2, 3 are configured to receive signals from controller 4, which may be software-based, to establish a remote center of motion at any suitable location.

Robotic surgical system 1 is configured for use on a patient “P” lying on a patient table 12 to be treated in a minimally invasive manner by an end effector of one or more of the surgical instruments. Surgical system 1 may also include more than two robotic arms 2, 3, the additional robotic arms likewise being connected to control device 4 and being telemanipulatable by operating console 5. One or more additional surgical assemblies 100 and/or surgical instruments 200, 300 may also be attached to the additional robotic arm.

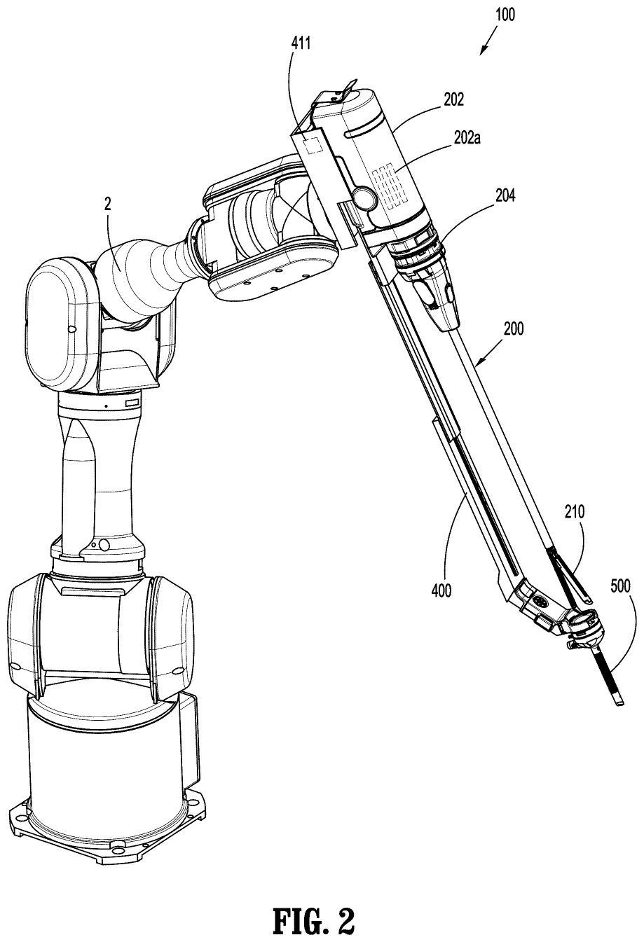

Control device 4 may control a plurality of motors (Motor 1 . . . n) with each motor configured to drive a pushing or a pulling of one or more cables of surgical instruments 200, 300. The plurality of motors can include a plurality of motors 202a of an instrument drive unit 202 that operate surgical instruments 200, 300 through a sterile interface module 204 when a respective one of surgical instruments 200, 300 is connected to sterile interface module 204 as shown in FIG. 2. In use, as these cables are pushed and/or pulled, the one or more cables effect operation and/or movement of end effectors 210, 310 of surgical instruments 200, 300. It is contemplated that control device 4 coordinates the activation of the various motors (Motor 1 . . . n) to coordinate a pushing or a pulling motion of these cables in order to coordinate an operation and/or movement of end effectors 210, 310. In aspects, each motor can be configured to actuate a drive rod or a lever arm to effect operation and/or movement of end effectors 210, 310 in addition to, or instead of one or more cables.

Control device 4 can include any suitable logic control circuit adapted to perform calculations and/or operate according to a set of instructions. Control device 4 can be configured to communicate with a remote system “RS,” either via a wireless (e.g., Wi-Fi, Bluetooth, LTE, etc.) and/or wired connection. Remote system “RS” can include data, instructions and/or information related to the various components, algorithms, and/or operations of workstation 1. Remote system “RS” can include any suitable electronic service, database, platform, cloud “C,” or the like. Control device 4 may include a central processing unit operably connected to memory. The memory may include transitory type memory (e.g., RAM) and/or non-transitory type memory (e.g., flash media, disk media, etc.). In some aspects, the memory is part of, and/or operably coupled to, remote system “RS.”

Control device 4 can include a plurality of inputs and outputs for interfacing with the components of robotic surgical system 1, such as through a driver circuit. Control device 4 can be configured to receive input signals and/or generate output signals to control one or more of the various components (e.g., one or more motors) of robotic surgical system 1. The output signals can include, and/or can be based upon, algorithmic instructions which may be pre-programmed and/or input by a user. Control device 4 can be configured to accept a plurality of user inputs from a user interface (e.g., switches, buttons, touch screen, etc. of operating console 5) which may be coupled to remote system “RS.”

A database 14 can be directly and/or indirectly coupled to control device 4. Database 14 can be configured to store pre-operative data from living being(s) and/or anatomical atlas(es). Database 14 can include memory which can be part of, and/or or operatively coupled to, remote system “RS.”

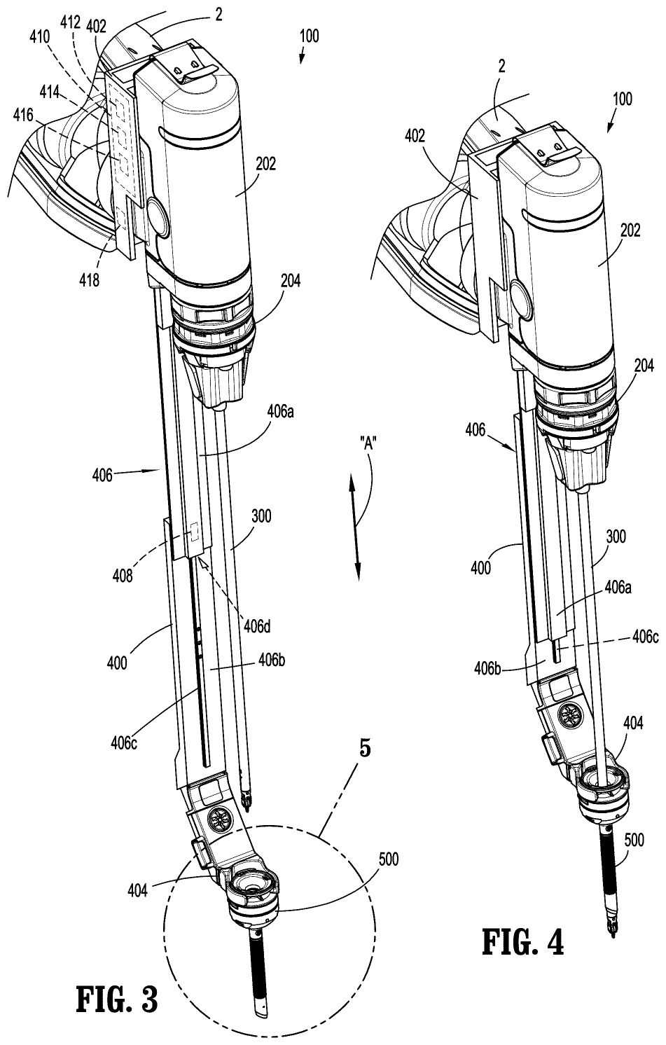

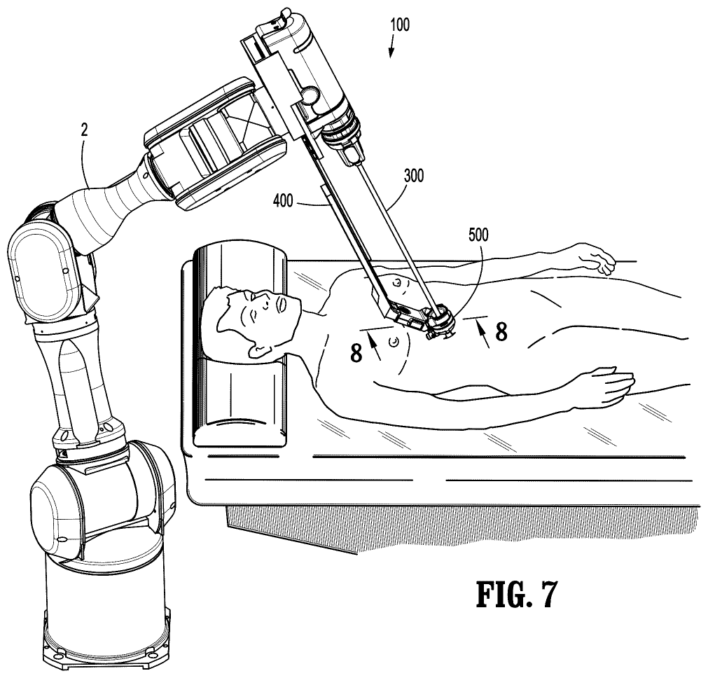

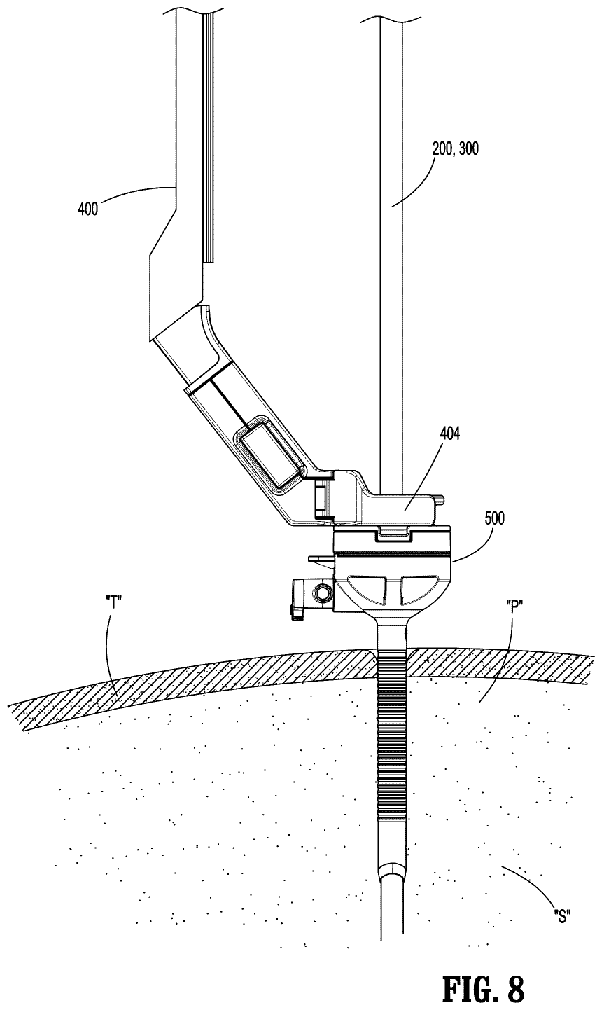

Turning now to FIGS. 3-8, surgical assembly 100 of robotic surgical system 1 includes an instrument drive unit 202, a sterile interface module 204 coupled to instrument drive unit 202, and one or more surgical instruments, such as surgical instrument 300, that are selectively attachable to sterile interface module 204 and actuatable by instrument drive unit 202.

Surgical assembly 100 further includes a linear slide mechanism 400 for actively supporting a surgical portal 500 on a distal end portion thereof. Linear slide mechanism 400 can be an active servo joint that provides redundant instrument insertion and retraction. In aspects, the linear slide mechanism 400, and/or components thereof, can include alloy material, composite material, and/or carbon fiber.

Linear slide mechanism 400 of surgical assembly 100 includes a drive housing 402 on a proximal end portion thereof that is coupled to instrument drive unit 202, and a mount assembly 404 on a distal end portion thereof that is configured to selectively couple to surgical portal 500 for securing surgical portal 500 to linear slide mechanism 400. Linear slide mechanism 400 further includes an arm assembly 406 that connects mount assembly 404 to drive housing 402 of linear slide mechanism 400. Arm assembly 406 is telescopic and includes a stationary segment 406a that extends distally from drive housing 402 and a movable segment 406b that is positioned to move in a linear direction (e.g., slide in a vertical direction) relative to stationary segment 406a, as illustrated by arrows “A,” to actively control a positioning of surgical portal 500 relative to a patient “P” and/or surgical instrument 300. Arm assembly 406 further includes an elongated rib 406c that extends along movable segment 406b that slides through a rib channel 406d defined in stationary segment 406a to facilitate sliding movement of movable segment 406b relative to stationary segment 406a. To facilitate linear or telescopic movement of arm assembly 406 of linear slide mechanism 400, linear slide mechanism 400 can include any suitable bearing or roller member 408 which may include, for example, a cross-roller, a cross-ball, and/or axial-radial roller and/or ball bearings, and/or any other suitable rolling/recirculating bearing and/or slider members. In aspects, linear slide mechanism 400 can include a slip clutch and/or force limiting sensor 411 (see FIG. 2) for facilitating robo-lift.

Linear slide mechanism 400 of surgical assembly 100 further includes a drive assembly 410 that supports a drive motor 412 (e.g., a servo actuator) and a drive 414 (e.g., a cable drive, belt drive, and/or a rack and pinion drive) that is actuated by drive motor 412 and which drives sliding movement of movable segment 406b relative to stationary segment 406a. In aspects, actuator 412 may include an integrated brake.

Linear slide mechanism 400 of surgical assembly 100 also includes an encoder 416, such as a linear encoder and/or a rotary encoder with linear-to-rotary transmission, that are operably coupled to drive housing 402 of linear slide mechanism 400 to monitor positions of movable segment 406b relative to stationary segment 406a. The linear slide mechanism 400 may include a rotary encoder at a drive motor, a cable pulley, a belt pulley, a pinion gear or linear encoder at the movable segments, a belt, a cable, a linear motor, or combinations thereof.

In aspects, the linear slide mechanism 400 can include a separate or independent brake 418 to hold any position through drive motor 412 or such independent brake 418. Drive motor 412 or such independent brake 418 may use a solenoid actuated plunger and/or friction holding of drive 414 of liner slide mechanism 400.

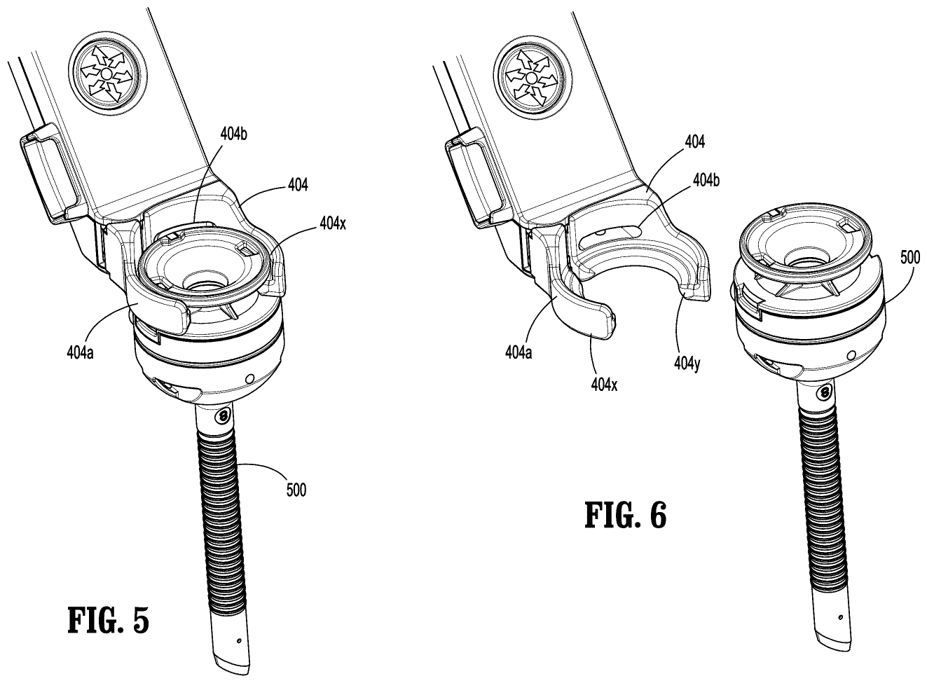

With reference to FIGS. 5 and 6, mount assembly 404 of linear slide mechanism 400 is supported on a distal end portion of movable segment 406b and is configured to removably support and/or hold surgical portal 500 thereon. This eliminates the need for an instrument to support the surgical portal 500 and prevents side loads on the instrument shafts to sense and monitor software RCM. Mount assembly 404 can be configured to facilitate detachment or attachment of surgical portal 500 with a large compliant ring 404x that extends around surgical portal 500 to reduce pressure on a patient's access position and which can be configured for locking operations or free mode operations. Compliant ring 404x of mount assembly 404 includes a manual and/or electronic clamp 404a. Manual and/or electronic clamp 404a of mount assembly 404 can be configured to rotationally and/or axially lock surgical portal 500 in position on mount assembly 404 and/or enable free rotation of surgical portal 500 with respect to mount assembly 404. Mount assembly 404 may include detectors 404b such as detection sensors, switches, and/or combinations thereof that detect the presence/absence of surgical portal 500 and/or axial and/or rotational movement of surgical portal 500 relative to mount assembly 404.

In aspects, compliant ring 404x may include an inflatable tube 404y that can be inflated or deflated as desired to maintain different predetermined pressures on surgical portal 500.

In use, as illustrated in FIGS. 1-8, for example, during a laparoscopic procedure, in which an abdominal region of a patient is insufflated to create a working space at the surgical site “S” (although the presently described surgical system can be used in any suitable open or minimally invasive procedure), surgical portal 500 is coupled to mount assembly 404 of linear slide mechanism 400. Utilizing the mechanical connection to surgical portal 500 and software based instructions to establish the remote center of motion (RCM), robotic arm 2 can be moved toward patient “P” and/or movable segment 406b of linear slide mechanism 400 can be moved relative to stationary segment 406a (e.g., extended distally) to positioned surgical portal 500 in tissue “T.” Further, with a surgical instrument such as surgical instrument 300 secured to sterile interface module 204, robotic arm 2 and/or movable segment 406b of linear slide mechanism 400 can be moved relative to one another to enable surgical instrument 300 to advance through surgical portal 500 for accessing the surgical site “S” utilizing the mechanical and software-based RCM. Positioning of movable segment 406b and/or robotic arm 2 can be adjusted (e.g., actively manipulated) as desired to reposition surgical instrument 300 and/or surgical portal 500 relative to patient “P” for reducing or limiting forces acting on the tissue “T” of the patient “P” through surgical portal 500 while effectuating a minimally invasive surgical procedure on the patient “P.” Moreover, robotic arm 2 and/or movable segment 406b of linear slide mechanism 400 can be moved relative to one another to enable instrument exchange and/or reinsertion/removal of the respective surgical instruments from surgical portal 500 as desired.

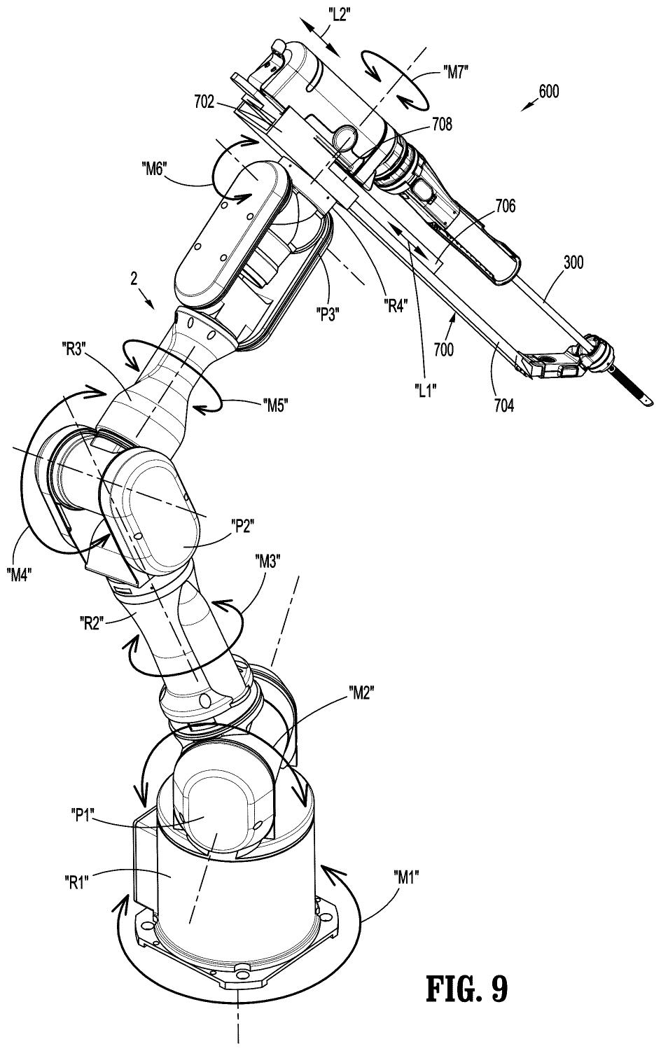

With reference to FIG. 9, surgical assembly 600 is similar to surgical assembly 100, but surgical assembly 600 includes a linear mechanism 700 instead of linear mechanism 400. Linear mechanism 700 includes a drive housing 702 that supports a fixed arm assembly 704. Fixed arm assembly 704 supports a movable carriage 706 thereon, that is configured to translate along fixed arm assembly 704, as indicated by arrows “L1.” Further, movable carriage 706 supports a movable rail 708 that is positioned to translate relative to moveable carriage 706, as indicated by arrows “L2,” to enable, for example, surgical instrument 300 to move relative to movable carriage 706. As can be appreciated, such linear movement can be effectuated simultaneously with and/or independently of movement of one or more of the joints of robotic arm 2. For instance, the various joints of robotic arm 2 can pivot and/or rotate in various directions as illustrated by the various arrows “M1” through “M7.” More specifically, robot arm 2 can include joint “R1”, joint “P1”, joint “R2”, joint “P2”, joint “R3”, and joint “P3”. In this regard, robot arm configurations (e.g., degrees of freedom) can include, for example, option 1: 1R1-2P1-3P2-4R2-5P3-6R3-7L1-8L2; option 2: 1R1-2P1-3R2-4P2-5R3-6P3-7R4-8L1-9L2; option 3: 1R1-2P1-3P2-4R2-5P3-6R3-7L1; and/or option 4: 1R1-2P1-3R2-4P2-5R3-6P3-7R4-8L1; where R connotes a revolute roll, P connotes a revolute pitch, L connotes a linear/prismatic movement, and where XRY or XPY refers to X being the first joint of the serial chain robotic arm and Y being the roll/pitch joint order number (starting from the first joint).

In aspects, fixed arm assembly 704 may have a telescope arrangement that may be actively controlled via software to effectuate linear movement “L1”. In aspects, linear movement indicated by “L2” may include independent software controlled active or passive joints that may provide manual instrument insertion and/or retraction. During manual mode, a latch holding the instrument position to the slide rail may be unlatched to enable the instrument to move relative to movable carriage 706.

In aspects, the instrument drive may include 1-axis instrument roll and/or 4-axis instrument drive.

In aspects, a force/torque sensor is included on the trocar and/or the port latch.

In aspects, there may be wireless communication between the instrument and the robotic arm.

It should be understood that the disclosed structure can include any suitable mechanical, electrical, and/or chemical components for operating the disclosed system or components thereof. For instance, such electrical components can include, for example, any suitable electrical and/or electromechanical, and/or electrochemical circuitry, which may include or be coupled to one or more printed circuit boards. As appreciated, the disclosed computing or control devices (and/or servers), can include, for example, a “controller,” “processor,” “digital processing device” and like terms, and which are used to indicate a microprocessor or central processing unit (CPU). The CPU is the electronic circuitry within a computer that carries out the instructions of a computer program by performing the basic arithmetic, logical, control and input/output (I/O) operations specified by the instructions, and by way of non-limiting examples, include server computers. In some aspects, the controller includes an operating system configured to perform executable instructions. The operating system is, for example, software, including programs and data, which manages hardware of the disclosed apparatus and provides services for execution of applications for use with the disclosed apparatus. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. In some aspects, the operating system is provided by cloud computing.

In some aspects, the term “controller” may be used to indicate a device that controls the transfer of data from a computer or computing device to a peripheral or separate device and vice versa, and/or a mechanical and/or electromechanical device (e.g., a lever, knob, etc.) that mechanically operates and/or actuates a peripheral or separate device.

In aspects, the controller includes a storage and/or memory device. The storage and/or memory device is one or more physical apparatus used to store data or programs on a temporary or permanent basis. In some aspects, the controller includes volatile memory and requires power to maintain stored information. In various aspects, the controller includes non-volatile memory and retains stored information when it is not powered. In some aspects, the non-volatile memory includes flash memory. In certain aspects, the non-volatile memory includes dynamic random-access memory (DRAM). In some aspects, the non-volatile memory includes ferroelectric random-access memory (FRAM). In various aspects, the non-volatile memory includes phase-change random access memory (PRAM). In certain aspects, the controller is a storage device including, by way of non-limiting examples, CD-ROMs, DVDs, flash memory devices, magnetic disk drives, magnetic tapes drives, optical disk drives, and cloud-computing-based storage. In various aspects, the storage and/or memory device is a combination of devices such as those disclosed herein.

In various aspects, the memory can be random access memory, read-only memory, magnetic disk memory, solid state memory, optical disc memory, and/or another type of memory. In various aspects, the memory can be separate from the controller and can communicate with the processor through communication buses of a circuit board and/or through communication cables such as serial ATA cables or other types of cables. The memory includes computer-readable instructions that are executable by the processor to operate the controller. In various aspects, the controller may include a wireless network interface to communicate with other computers or a server. In aspects, a storage device may be used for storing data. In various aspects, the processor may be, for example, without limitation, a digital signal processor, a microprocessor, an ASIC, a graphics processing unit (“GPU”), field-programmable gate array (“FPGA”), or a central processing unit (“CPU”).

The memory stores suitable instructions and/or applications, to be executed by the processor, for receiving the sensed data (e.g., sensed data from camera), accessing storage device of the controller, generating a raw image based on the sensed data, comparing the raw image to a calibration data set, identifying an object based on the raw image compared to the calibration data set, transmitting object data to a post-processing unit, and displaying the object data to a graphic user interface. Although illustrated as part of the disclosed structure, it is also contemplated that a controller may be remote from the disclosed structure (e.g., on a remote server), and accessible by the disclosed structure via a wired or wireless connection. In aspects where the controller is remote, it is contemplated that the controller may be accessible by, and connected to, multiple structures and/or components of the disclosed system.

The term “application” may include a computer program designed to perform particular functions, tasks, or activities for the benefit of a user. Application may refer to, for example, software running locally or remotely, as a standalone program or in a web browser, or other software which would be understood by one skilled in the art to be an application. An application may run on the disclosed controllers or on a user device, including for example, on a mobile device, an IOT device, or a server system.

In some aspects, the controller includes a display to send visual information to a user. In various aspects, the display is a cathode ray tube (CRT). In various aspects, the display is a liquid crystal display (LCD). In certain aspects, the display is a thin film transistor liquid crystal display (TFT-LCD). In aspects, the display is an organic light emitting diode (OLED) display. In certain aspects, on OLED display is a passive-matrix OLED (PMOLED) or active-matrix OLED (AMOLED) display. In aspects, the display is a plasma display. In certain aspects, the display is a video projector. In various aspects, the display is interactive (e.g., having a touch screen) that can detect user interactions/gestures/responses and the like. In some aspects, the display is a combination of devices such as those disclosed herein.

The controller may include or be coupled to a server and/or a network. As used herein, the term “server” includes “computer server,” “central server,” “main server,” and like terms to indicate a computer or device on a network that manages the disclosed apparatus, components thereof, and/or resources thereof. As used herein, the term “network” can include any network technology including, for instance, a cellular data network, a wired network, a fiber-optic network, a satellite network, and/or an IEEE 802.11a/b/g/n/ac wireless network, among others.

In various aspects, the controller can be coupled to a mesh network. As used herein, a “mesh network” is a network topology in which each node relays data for the network. All mesh nodes cooperate in the distribution of data in the network. It can be applied to both wired and wireless networks. Wireless mesh networks can be considered a type of “Wireless ad hoc” network. Thus, wireless mesh networks are closely related to Mobile ad hoc networks (MANETs). Although MANETs are not restricted to a specific mesh network topology, Wireless ad hoc networks or MANETs can take any form of network topology. Mesh networks can relay messages using either a flooding technique or a routing technique. With routing, the message is propagated along a path by hopping from node to node until it reaches its destination. To ensure that all its paths are available, the network must allow for continuous connections and must reconfigure itself around broken paths, using self-healing algorithms such as Shortest Path Bridging. Self-healing allows a routing-based network to operate when a node breaks down or when a connection becomes unreliable. As a result, the network is typically quite reliable, as there is often more than one path between a source and a destination in the network. This concept can also apply to wired networks and to software interaction. A mesh network whose nodes are all connected to each other is a fully connected network.

In some aspects, the controller may include one or more modules. As used herein, the term “module” and like terms are used to indicate a self-contained hardware component of the central server, which in turn includes software modules. In software, a module is a part of a program. Programs are composed of one or more independently developed modules that are not combined until the program is linked. A single module can contain one or several routines, or sections of programs that perform a particular task.

As used herein, the controller includes software modules for managing various aspects and functions of the disclosed system or components thereof.

The disclosed structure may also utilize one or more controllers to receive various information and transform the received information to generate an output. The controller may include any type of computing device, computational circuit, or any type of processor or processing circuit capable of executing a series of instructions that are stored in memory. The controller may include multiple processors and/or multicore central processing units (CPUs) and may include any type of processor, such as a microprocessor, digital signal processor, microcontroller, programmable logic device (PLD), field programmable gate array (FPGA), or the like. The controller may also include a memory to store data and/or instructions that, when executed by the one or more processors, cause the one or more processors to perform one or more methods and/or algorithms.

The phrases “in an aspect,” “in aspects,” “in various aspects,” “in some aspects,” or “in other aspects” may each refer to one or more of the same or different aspects in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques).

Certain aspects of this disclosure may include some, all, or none of the above advantages and/or one or more other advantages readily apparent to those skilled in the art from the drawings, descriptions, and claims included herein. Moreover, while specific advantages have been enumerated above, the various aspects of the present disclosure may include all, some, or none of the enumerated advantages and/or other advantages not specifically enumerated above.

The aspects disclosed herein are examples of the disclosure and may be embodied in various forms. For instance, although certain aspects herein are described as separate, each of the aspects herein may be combined with one or more of the other aspects herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

Any of the herein described methods, programs, algorithms, or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

As can be appreciated, securement of any of the components of the disclosed systems can be effectuated using known securement techniques such welding, crimping, gluing, fastening, etc.

Persons skilled in the art will understand that the structures and methods specifically described herein and shown in the accompanying figures are non-limiting exemplary aspects, and that the description, disclosure, and figures should be construed merely as exemplary of particular aspects. It is to be understood, therefore, that the present disclosure is not limited to the precise aspects described, and that various other changes and modifications may be effected by one skilled in the art without departing from the scope or spirit of the disclosure. Additionally, the elements and features shown or described in connection with certain aspects may be combined with the elements and features of certain other aspects without departing from the scope of the present disclosure, and that such modifications and variations are also included within the scope of this disclosure. Accordingly, the subject matter of this disclosure is not limited by what has been particularly shown and described.

What is claimed:

1. A robotic surgical system, comprising:
a robotic arm;
a surgical instrument;
a surgical portal; and
a linear slide mechanism coupled to the robotic arm and configured to movably support the surgical portal relative to the surgical instrument.

2. The robotic surgical system of claim 1, wherein the linear slide mechanism includes an arm assembly including a stationary segment and a movable segment, the movable segment positioned to move relative to the stationary segment.

3. The robotic surgical system of claim 2, wherein the movable segment is slides along the stationary segment.

4. The robotic surgical system of claim 3, wherein the linear slide mechanism includes a drive housing on a proximal end portion thereof, the drive housing coupled to the robotic arm, the stationary segment extending from the drive housing.

5. The robotic surgical system of claim 4, wherein the drive housing is coupled to an instrument drive unit that operates the surgical instrument.

6. The robotic surgical system of claim 5, further comprising a sterile interface module that connects the surgical instrument to the instrument drive unit.

7. The robotic surgical system of claim 6, wherein the movable segment is movable relative to the surgical instrument.

8. The robotic surgical system of claim 7, wherein the movable segment supports a mount assembly that is configured to support the surgical portal.

9. The robotic surgical system of claim 8, wherein the drive housing supports a drive motor and a drive that is operable by the drive motor to cause the movable segment to move relative to the stationary segment.

10. The robotic surgical system of claim 9, wherein the drive includes a cable drive, a belt drive, a rack and pinion drive, electromagnetic linear drive, or combinations thereof.

11. A surgical system, comprising:
an instrument drive unit;
a surgical instrument coupled to the instrument drive unit;
a surgical portal; and
a linear slide mechanism configured to movably support the surgical portal relative to the surgical instrument.

12. The surgical system of claim 11, wherein the linear slide mechanism includes an arm assembly including a stationary segment and a movable segment, the movable segment positioned to move relative to the stationary segment.

13. The surgical system of claim 12, wherein the movable segment slides along stationary segment.

14. The surgical system of claim 13, wherein the linear slide mechanism includes a drive housing on a proximal end portion thereof, the drive housing coupled to the instrument drive unit.

15. The surgical system of claim 14, wherein the drive housing supports an encoder configured to monitor positions of the movable segment relative to the stationary segment.

16. The surgical system of claim 15, further comprising a sterile interface module that connects the surgical instrument to the instrument drive unit.

17. The surgical system of claim 16, wherein the movable segment is movable relative to the surgical instrument.

18. The surgical system of claim 17, wherein the movable segment supports a mount assembly that is configured to support the surgical portal.

19. The surgical system of claim 18, wherein the drive housing supports a drive motor and a drive that is operable by the drive motor to cause the movable segment to move relative to the stationary segment.

20. A robotic surgical system, comprising:
a robotic arm;
an instrument drive unit;
a surgical instrument removably coupled to the instrument drive unit;
a surgical portal;
a controller; and
a linear slide mechanism positioned between the robotic arm and the instrument drive unit, the linear slide mechanism including a movable segment that is operatively coupled to the controller to enable the controller to actively move the surgical portal relative to a patient.
